(19) United States
(12) Patent Application Publication
(10) Pub. No.: US 2010/0016951 A1
(43) Pub. Date: Jan. 21, 2010

(54) STENT

(76) Inventors: Young-Shin Kim, Pyeongtaek (KR); Seong-Hyeon Kim, Pyeongtaek (KR); Hyun-Joo Kim, Pyeongtaek-si (KR)

Correspondence Address: GIFFORD, KRASS, SPRINKLE,ANDERSON & CITKOWSKI, P.C, PO BOX 7021, TROY, MI, 48007-7021, US

(21) Appl. No.: 12/373,808

(22) PCT Filed: Mar. 26, 2007
(86) PCT No.: PCT/KR2007/001461

§ 371 Date: Jan. 14, 2009

(30) Foreign Application Priority Data
Jul. 20, 2006 (KR) 1020060068012

(51) Int. Cl. A61F 2/06

(52) U.S. Cl. 623/1.16

(57) ABSTRACT

A stent for securing a passageway of a body lumen in a constricted portion of the body lumen is disclosed. The stent includes a first cylindrical band body formed in a zigzag shape having a plurality of straight sections interconnected by first peaks and first valleys and having opposite ends that are integrally formed, a second cylindrical band body having a same structure as the first cylindrical band body, the second cylindrical band body being disposed such that second valleys of the second cylindrical band body face the respective first peaks of the first cylindrical band body and second peaks of the second cylindrical band body face the respective first valleys of the first cylindrical band body with a plurality of predetermined gaps of a first row, thereby defining a plurality of rhombus spaces of a first row, at least one first link member that is provided in at least one rhombus spaces of the first row to interconnect the first and second band bodies, at least one second link members that are provided in at least one rhombus spaces of the second row to interconnect the second and third band bodies.

### TECHNICAL FIELD

The present invention relates to a stent. More particularly, the present invention relates to a stent that is inserted in a body lumen such as a blood vessel, an esophagus, and the like to counteract significant decrease in a lumen diameter and thus to maintain an adequate delivery of blood or food.

### BACKGROUND ART

When stenosis occurs in a body lumen such as a blood vessel or an esophagus due to a congenital or acquired disease, blood or food cannot be effectively delivered through corresponding body lumens. In this case, an expandable device that is referred to as stent is inserted in the body lumen to increase a diameter of the passageway of the body lumen. This is a well-known technology in the art.

A catheter is used to insert the stent in a stenosis portion of the body lumen. The stent inserted in the stenosis portion is configured to expand the stenosis portion by self-elastic force.

The stent is generally formed of a metal wire and provided in the form of a perforated elastic tube shape. However, a conventional fabrication method of such a stent is complicated and has a limit in fabricating a small-sized stent. Therefore, in recent years, the stent has been fabricated by processing a thin metal cylindrical body using a laser beam. This stent is configured to be elastically self-expandable in a redial direction from a contracted state.

The stent is processed by the laser beam such that it includes at least two cylindrical band bodies arranged and interconnected lengthwise. Each of the cylindrical band bodies is formed in a zigzag shape having a plurality of valleys and a plurality of peaks that are alternately arranged along a circumferential direction.

With the above-described structure where the band bodies are arranged lengthwise, the valleys of one of the band bodies are arranged to correspond to the valleys of adjacent one of the band bodies and the peaks of one of the band bodies are arranged to correspond to the peaks of adjacent one of the band bodies.

Links are connected between the band bodies so that the band bodies define the cylindrical body shape.

In the above-described conventional stent, the links are connected between the valleys of the adjacent band bodies and between the peaks of the adjacent band bodies.

The conventional stent has a limit in increasing a connecting section of the links as compared with a gap between the band bodies. When a length of the link is short, the flexibility of the stent is deteriorated,

Further, when the conventional stent is inserted in a curved body lumen, a phenomenon where the adjacent valleys and adjacent peaks overlap with each other or protrudes to a central portion of the curved body lumen at the connection section of the band bodies. Particularly, the protrusion of the valleys and peaks toward the central portion of the body lumen causes the blocking the passageway of the body lumen in which the stent is inserted. This may be fatal to the human body.

### DISCLOSURE

### Technical Problem

The present invention has been made in an effort to solve the above-described problems of the prior art. Exemplary embodiments of the present invention provide a stent that is inserted in a stenosis portion of a body lumen to counteract significant decrease in a passageway diameter of the body lumen.

### Technical Solution

According to an exemplary embodiment of the present invention, a stent includes:

a first cylindrical band body formed in a zigzag shape having a plurality of straight sections interconnected by first peaks and first valleys and having opposite ends that are integrally formed;

a second cylindrical band body having a same structure as the first cylindrical band body, the second cylindrical band body being disposed such that second valleys of the second cylindrical band body face the respective first peaks of the first cylindrical band body and second peaks of the second cylindrical band body face the respective first valleys of the first cylindrical band body with a plurality of predetermined gaps of a first row, thereby defining a plurality of rhombus spaces of a first row;

a third cylindrical band body having a same structure as the first cylindrical band body, the third cylindrical band body being disposed such that third valleys of the third cylindrical band body face the respective second peaks of the second cylindrical band body and third peaks of the second cylindrical band body face the respective second valleys of the second cylindrical band body with a plurality of predetermined gaps of a second row, thereby defining a plurality of rhombus spaces of a second row;

at least one first link member that is provided in at least one rhombus spaces of the first row to interconnect the first and second band bodies; and

at least one second link members that are provided in at least one rhombus spaces of the second row to interconnect the second and third band bodies.

The first link members may be alternately arranged with the second link members.

The stent may be formed of a shape memory alloy.

The shape memory alloy may be nitinol.

The stent may further include one or more additional cylindrical band bodies that are arranged lengthwise of the stent and interconnected by additional link members and one of the additional cylindrical bodies is connected to one of the first and third cylindrical band bodies.

### Advantageous Effects

According to the stent of the present invention, since the band bodies of the stent are interconnected by first and second link members having different lengths, the flexibility of the stent can be improved and thus the stent of the present invention effectively prevents the blocking of the lumen.

Particularly, even when the stent is inserted in the lumen whose curvature is relatively high, the deformation of the stent can be minimized.

### DESCRIPTION OF DRAWINGS

FIG. 1 is a schematic perspective view of a stent according to a first exemplary embodiment of the present invention.

FIG. 2 is a developed diagram of the stent of FIG. 1.

FIG. 3 illustrates an application of the stent of FIG. 1 to a body lumen.

FIG. 4 illustrates an operation of a first link of the stent of FIG. 1.

FIG. 5 illustrates an operation of a second link of the stent of FIG. 1.

FIG. 6 is a developed diagram of a stent according to a second exemplary embodiment of the present invention.

FIG. 7 illustrates an application of the stent of FIG. 6 to a body lumen.

### BEST MODE

Exemplary embodiments of the present invention will now be described with reference to the accompanying drawings. The following exemplary embodiments will be described within a range where persons having ordinary skill in the art can understand and implement.

The invention may, however, be embodied in many different forms and should not be construed as being limited to the exemplary embodiments set forth herein.

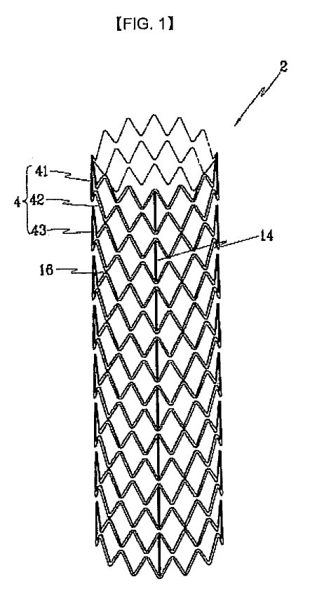

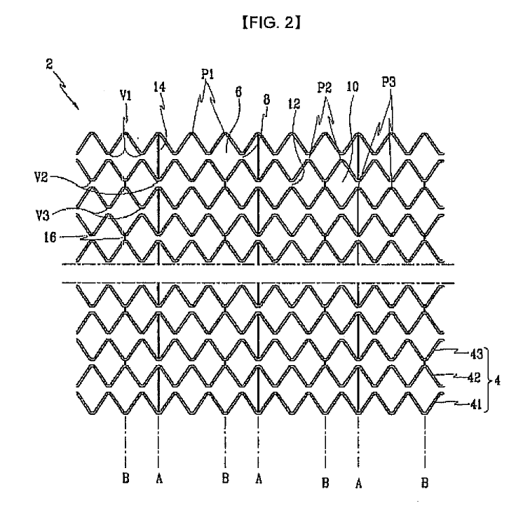

FIG. 1 is a perspective view of a stent according to a first exemplary embodiment of the present invention and FIG. 2 is a developed view of the stent of FIG. 1.

A stent 2 of an exemplary embodiment of the present invention includes two or more cylindrical band bodies 4 that are arranged and spaced apart from each other along a central axis C thereof, thereby defining a thin, long cylindrical elastic body.

The number of the cylindrical band bodies may vary depending on a lo use of the stent.

The cylindrical band bodies 4 include a first cylindrical band body 41, a second cylindrical band body 42, and a third cylindrical band body 43. Structures of the first, second, and third cylindrical band bodies 41, 42, and 43 are identical to each other.

That is, the cylindrical band body 41, 42, 43 has a plurality of peaks P1, P2, P3 and a plurality of valleys V1, V2, V3. The peaks of each of the cylindrical band bodies 41, 42, and 43 are spaced apart from each other in a circumferential direction by a uniform distance and the valleys of each of the cylindrical band bodies 41, 42, and 43 are also spaced apart from each other in the circumferential direction at a uniform distance, thereby defining a cylindrical body shape. The cylindrical band bodies 41, 42, and 43 are fabricated by processing a thin cylindrical plate through a well-known laser process technology. The thin cylindrical plate may be formed of a shape memory alloy such as nitnol.

With the stent having the above-described structure, connecting members are provided to connect the peaks of one of the cylindrical band bodies 41, 42, and 43 to the valleys of adjacent one of the cylindrical band bodies 42, 42 and 43.

The first cylindrical band body 41 is formed in a zigzag shape having a plurality of straight sections. The adjacent straight sections are interconnected by the peaks P1 and the valleys V1. Opposite ends of the cylindrical band body 41 are integrally formed with each other, thereby defining the cylindrical shape.

The second cylindrical band body 42 is formed in the same shape as the first cylindrical band body 41. At this point, the second cylindrical band body 42 is disposed such that the valleys V2 of the second cylindrical band body 42 face the respective peaks P1 of the first cylindrical band body 41 and the peaks P2 of the second cylindrical band body 42 face the respective valleys V1 of the first cylindrical band body 43 with a plurality of predetermined gaps 8 of a first row, thereby defining a plurality of rhombus spaces 6 of a first row.

The third cylindrical band body 43 is formed in the same shape as the first cylindrical band body 41. At this point, the third cylindrical band body 43 is disposed such that the valleys V3 of the third cylindrical band body 43 face the respective peaks P2 of the second cylindrical band body 42 and the peaks P3 of the third cylindrical band body 43 face the respective valleys V2 of the second cylindrical band body 42 with predetermined gaps 12 of a second row, thereby defining a plurality of rhombus spaces 10 of a second row. The gaps of the first row and the gaps of the second row are aligned along diagonal lines.

The connecting members provided between the peaks and the valleys of the adjacent cylindrical band bodies includes first links 14 that are disposed in at least two rhombus spaces 6 of the first row to interconnect the first and second cylindrical band bodies 41 and 42.

The connecting members further include one or more second link members 16 that are disposed in at least one of the gaps 12 of the second row to connect the second cylindrical band body 42 to the third cylindrical band body 43.

The first link members 14 are provided in at least two rhombus spaces 6 of the first row and spaced apart from each other in a circumferential direction of the cylindrical band bodies 4. The link members 14 may be integrally formed with the cylindrical band bodies 4 or attached to the cylindrical band bodies 4 by welding.

The link members 14 and 16 provides flexibility between the band bodies 4 so as to prevent the band bodies from being deformed toward the central axis of the stent when the stent is bent in a direction.

That is, in the present exemplary embodiment, the first link members 14 function to improve overall flexibility of the stent and the second link members 16 function to prevent the deformation of the stent.

As described above, additional cylindrical band bodies may be further arranged along the central axis C of the stent. Therefore, the first line member 14 are disposed lengthwise of the cylindrical band body.

At this point, as shown in FIGS. 1 and 2, the link members 14 of the different cylindrical band bodies 4 are arranged along common lines.

A length of the second link member 14 is less than that of the first link member 16 so as to minimize the twist between the cylindrical band bodies 4.

Two or more of the second link members 16 may be provided and spaced apart each other in the circumferential direction of each second line member 16. The second link members 16 are alternately arranged with the first link members 14 in the circumferential direction.

That is, as shown in FIG. 2, when the first link members 14 are arranged in a column A, the second link members 16 are arranged in a column B.

This arrangement of the link members 14 and 16 improves the flexibility and stiffness of the stent.

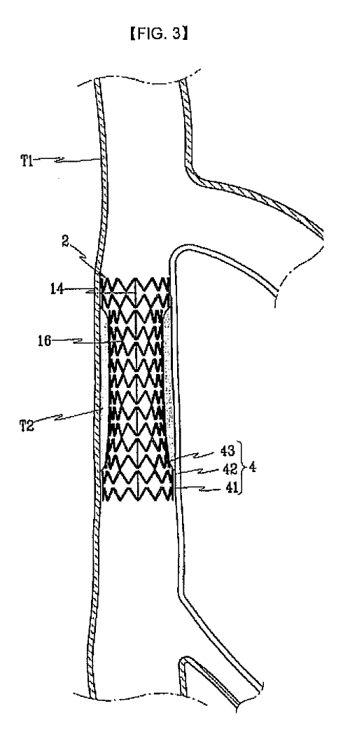

In the stent of the present exemplary embodiment of the present invention, since the first link members 14 are provided between the peaks and valleys of the respective adjacent band bodies 4, the flexibility of the stent is improved. Therefore, as shown in FIG. 3, when the stent 2 is inserted in a constricted portion of a body lumen T1 such as a blood vessel or an esophagus, the passageway can be effectively secured even at the constricted portion.

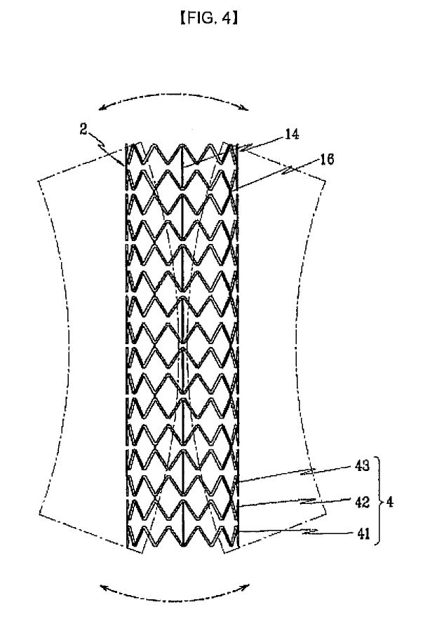

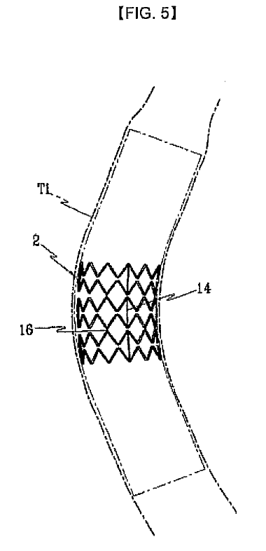

In addition, as shown in FIG. 4, the stent 2 can be flexibly bent in a radial direction. Therefore, even when the stent 2 is inserted in a curved body lumen T1 as shown in FIG. 5, it can effectively secure a passageway of the curve body lumen T1 by being inserted in the constricted portion of the curved body lumen T1.

Particularly, when a conventional stent is inserted in the curved body lumen T1, the connecting portions of the band bodies may protrude to a central portion of the body lumen. However, according to the present embodiment of the present invention, since the band bodies 4 of the stent 2 are interconnected by the second link members each having a relatively short length, the protrusion of the connecting portions of the band bodies to the central portion of the lumen can be prevented.

That is, the conventional stent is configured to receive force urging the connecting portion of the band bodies 4 to protrude toward the central portion is of the curved body lumen as the adjacent valleys and peaks overlap with each other. However, in the present exemplary embodiment of the present invention, since the second link members 16 are connected between the adjacent valleys and peaks, the second link members 16 resist against the force urging the protrusion of the connecting portion toward the central portion of the curve body lumen, thereby minimizing the deformation of the stent.

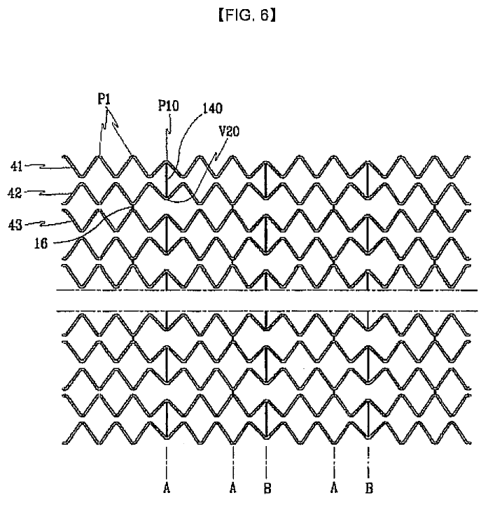

FIG. 6 is a developed view of a stent according to another exemplary embodiment of the present invention. Since a stent of this exemplary embodiment is similar to the stent of the foregoing embodiment, only different parts will be described hereinafter.

According to a stent of the present embodiment among a plurality of peaks of a first band body 41, heights of peaks P10 to which first link members 14 are connected are less than heights of other peaks P1. In addition, among a plurality of valleys of a second band body 42, depths of valleys V20 to which the first link members 14 are connected are less than depths of other valleys V2.

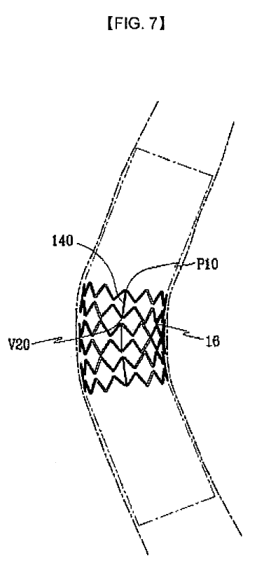

Accordingly, a length of the first link member 140 of the present exemplary embodiment becomes less than a length of the first link member of the foregoing exemplary embodiment of FIG. 2. Therefore, as shown in FIGS. 6 and 7, a gap between the adjacent first link members 14 arranged in a column A increases.

Accordingly, as shown in FIG. 7, even when the stent is inserted in a body lumen that is severely curved, the phenomenon where the connecting portion of band bodies 4 protrudes toward the central portion of the curved body lumen as the adjacent valleys and peaks overlap with each other.

### INDUSTRIAL APPLICABILITY

The stent of the present invention can be used to secure a passageway of a body lumen by being inserted in a constricted portion of the body lumen.

1. A stent comprising:
a first cylindrical band body formed in a zigzag shape having first peaks and first valleys and having opposite ends that are integrally formed;
a second cylindrical band body having the same structure as the first cylindrical band body, the second cylindrical band body being disposed such that second valleys of the second cylindrical band body face the respective first peaks of the first cylindrical band body and second peaks of the second cylindrical band body face the respective first valleys of the first cylindrical band body with a plurality of predetermined gaps of a first row, thereby defining a plurality of rhombus spaces of the first row;
a third cylindrical band body having a same structure as the first cylindrical band body, the third cylindrical band body being disposed such that third valleys of the third cylindrical band body face the respective second peaks of the second cylindrical band body and third peaks of the second cylindrical band body face the respective second valleys of the second cylindrical band body with a plurality of predetermined gaps of a second row, thereby defining a plurality of rhombus spaces of the second row in a diagonal direction with respect to the first row;
at least one first link member that is provided in at least one rhombus spaces of the first row to interconnect the first and second band bodies; and
at least one second link member that is provided in at least one rhombus spaces of the second row to interconnect the second and third band bodies.

2. The stent of claim 1, wherein the first link members are alternately arranged with the second link members.

3. The stent of claim 1, which is formed of a shape memory alloy,

4. The stent of claim 3, wherein the shape memory alloy is nitinol.

5. The stent of claim 1, further comprising one or more additional cylindrical band bodies that are arranged lengthwise of the stent and interconnected by additional link members and one of the additional cylindrical bodies is connected to one of the first and third cylindrical band bodies.

6. The stent of claim 1, wherein a height of each of the peaks of the first cylindrical band body is same as a depth of each of the valleys of the first cylindrical band body.

7. The stent of claim 1, wherein, among the peaks of the first cylindrical band body, at least two peaks have heights less than those of the rest of the peaks.

8. The stent of claim 7, wherein the link members are connected to the respective peaks having the less heights.

9. The stent of claim 1, wherein, among the valleys of the first cylindrical band body, at least two valleys have depths less than those of the rest of the valleys.
